(19) United States
(12) Patent Application Publication
(10) Pub. No.: US 2003/0103977 A1
(43) Pub. Date: Jun. 5, 2003

(54) Antibodies to polysaccharide of C. neoformans

(76) Inventors: Arturo Casadevall, Pelham, NY (US); Matthew D. Scharff, Larchmont, NY (US); Jean Mukherjee, Cedar Park, TX (US)

Correspondence Address: Elie H. Gendloff, Amster, Rothstein & Ebenstein, 90 Park Avenue, New York, NY, 10016, US

(21) Appl. No.: 10/254,198

(22) Filed: Sep. 24, 2002

Related U.S. Application Data
(63) Continuation of application No. 09/273,831, filed on Mar. 22, 1999, now abandoned.
Application No. 09/273,831 is a continuation of application No. 08/920,185, filed on Aug. 25, 1997, now abandoned.
Application No. 08/920,185 is a continuation of application No. 08/760,502, filed on Dec. 5, 1996, now abandoned.
Application No. 08/760,502 is a continuation of application No. 08/279,835, filed on Jul. 25, 1994, now abandoned.
Application No. 08/279,835 is a continuation of application No. 07/781,423, filed on Oct. 22, 1991, now abandoned.

(51) Int. Cl. A61K 39/395; C12P 21/04; C07K 16/20

(52) U.S. Cl. 424/151.1; 435/70.21; 530/388.6

(57) ABSTRACT

This invention relates to monoclonal antibodies which bind to non-enhancing protective epitopes on serotype A, B, C and D strains of C. neoformans, such protective epitopes containing acetyl groups in the polysaccharide of the epitopes. Other monoclonal antibodies of this invention are serotype specific, and bind to acetyl groups on polysaccharide capsule protective epitopes of serotype D strain C. neoformans only. This invention further relates to methods for producing these monoclonal antibodies. These monoclonal antibodies may be passively administered to treat and prevent cryptococcal infection, such as Cryptococcal meningitis, in immunosuppressed patients. These monoclonal antibodies may also be used for detection of fungal infection, for the development of diagnostic serotyping of clinical isolates, and as therapeutic adjuncts to anti-fungal antibiotic therapy.

### STATEMENT OF GOVERNMENT INTEREST

This invention was made with government support under NIH Grant Numbers CA09173 and CA39838. The government has certain rights in this invention.

### FIELD OF THE INVENTION

This invention relates to monoclonal antibodies which bind to protective epitopes on the polysaccharide capsule of serotype A, B, C and D strains of Cryptococcus neoformans, such protective epitopes containing acetyl groups in the polysaccharide thereof. Other monoclonal antibodies of this invention are serotype specific, and bind to protective epitopes on the polysaccharide capsule of serotype D C. neoformans only. This invention further relates to methods for producing these monoclonal antibodies. These antibodies may be used to treat cryptococcal infection, such as Cryoptococcal meningitis, especially in immunosuppressed patients, and may prevent cryptococcal infections by passive administration thereof. The monoclonal antibodies of this invention may be used for detection of fungal infection, development of diagnostic serotyping of clinical isolates, and as therapeutic adjuncts to anti-fungal antibiotic therapy.

### BACKGROUND OF THE INVENTION

Cryptococcus neoformans (C. neoformans) is a fungus which causes serious infection in humans. Immunocompromised individuals, such as AIDS patients, are at particular risk. C. neoformans causes disease in up to 10% of individuals with AIDS. In the setting of AIDS, cryptococcal infections are usually incurable and often fatal.

C. neoformans has a large polysaccharide capsule that inhibits phagocytosis by macrophages. The capsular polysaccharide is poorly immunogenic and causes the phenomenon of immune paralysis in mice. Structural differences in the capsular polysaccharides allow the grouping of cryptococcal strains into four serotypes, A, B, C and D. Most human disease is caused by strains of serotypes A and D.

Cellular immunity is believed to provide the primary host defense against C. neoformans. The role of humoral immunity to the C. neoformans capsular polysaccharide (CNPS) in protection is uncertain. It is likely that antibodies play an important role in the defense against C. neoformans because individuals with cryptococcal infection have a better prognosis if they have serum antibodies, as antibodies enhance phagocytosis by macrophages, mediate fungistasis by natural killer cells, and facilitate leukocyte killing. However, certain observations are not consistent with an important role for humoral immunity. For example, B-cell deficient mice are not especially susceptible to cryptococcal infection. In addition, vaccination with immunogenic polysaccharide glycoconjugates has not been protective in mice. Finally, no monoclonal antibodies to serotype A, B, C and D strains of C. neoformans have conferred protection after passive administration thereof. Several in vitro observations have indicated an important role for antibodies by enhancing cellular immunity, whereas some in vivo experiments have confirmed a protective effect and some have not. The finding that AIDS patients lack anti-CNPS IgG antibody raises the possibility that a lack of antibody contributes to their marked susceptibility to cryptococcus.

Monoclonal antibodies raised against CNPS have been generated by others using animals immunized with CNPS. See Dromer, F., Salamero, J., Contrepois, A., Carbon, C., and Yeni, P., “Production, Characterization and Antibody Specificity of a Mouse Monoclonal Antibody Reactive with Croptococcus neoformans Capsular Polysaccharide”, Infect. Immun. 55: 742-748 (1987); Dromer, F., Charreire, J., Contrepois, A., Carbon, C., and Yeni, P., “Protection of Mice Against Experimental Cryptococcus by Anti-Cryptococcus neoformans Monoclonal Antibody”, Infect Immun. 55: 749-752 (1987); Dromer, F. and Charreire, J., “Improved Amphotericin B (AMB) Activity by a Monoclonal Anti-Cryptococcus neoformans Antibody E₁ In Vivo and In Vitro Studies”, ICAAC Abstract #484 (1990); Eckert, T. F. and Kozel, T. R., “Production and Characterization of Monoclonal Antibodies Specific for Cryptococcus neoformans Capsular Polysaccharide”, Infect. Immun. 55: 1895-1899 (1987); Sanford, J., Lupan, D., Schlageter, A., and Kozel, T., “Passive Immunization against Cryptococcus neoformans with an Isotype-Switch Family of Monoclonal Antibodies Reactive with Cryptococcal Polysaccharide”, Infect. Immun. 58: 1919-1923 (1990) (wherein monoclonal antibodies to C. neoformans which were passively administered did not increase survival or confer protection); and Todaro-Luck, F., Reiss, E., Cherniak, R., and Kaufman, L., “Characterization of Cryptococcus neoformans Capsular Glucuronoxylomannan Polysaccharide with Monoclonal Antibodies,” Infect Immun. 57: 3882-3887 (1989).

Not all monoclonal antibodies to C. neoformans are protective. For example, Sanford et al. have described non-protective antibodies to C. neoformans. Further, some anti-cryptococcal antibodies can actually be deleterious in some circumstances. Such deleterious antibodies may be analogous to “enhancing” antibodies described in viral infections. Enhancing antibodies can arise during viral infections. These antibodies mediate disease enhancement by binding to viral particles, thereby facilitating entry of the virus into cells via Fc or complement receptors on the host cell surface. Uptake of the virus allows the particle to bypass its normal, perhaps more difficult route of host cell entry. This mechanism may create a greater cellular viral burden. In addition, infected host cells can transport the virus throughout the body resulting in invasion of distant or immunologically privileged areas such as the brain. This results in widespread dissemination and may accelerate disease.

In the case of cryptococcal infections, enhancing antibodies would increase the uptake of fungus by macrophages, but the fungus would not be killed. The macrophages would then circulate throughout the body, resulting in widespread fungal dissemination. This may be the mechanism by which C. neoformans migrates to the brain. Therefore, it is necessary that monoclonal antibodies used to treat cryptococcal infection not be enhancing antibodies.

The monoclonal antibodies of this invention are different from those described by others in that they are specific for non-enhancing protective epitopes on all four serotype A, B, C and D strains of C. neoformans, such epitopes containing acetyl groups in the polysaccharide. Other monoclonal antibodies of this invention, which are specific for serotype D strain C. neoformans only, also bind to protective epitopes on the polysaccharide of C. neoformans. In addition, the monoclonal antibodies of this invention were derived from B-cells stimulated during the response to infection with the actual C. neoformans organism or with a conjugate of the glucuronoxylomannan (a portion of the polysaccharide capsule of C. neoformans) to tetanus toxoid. The monoclonal antibodies may be used in the treatment and prevention of cryptococcus infection, and diminish the level of C. neoformans polysaccharide circulating in body fluids.

### SUMMARY OF THE INVENTION

This invention is directed to monoclonal antibodies which recognize non-enhancing protective epitopes on all four serotype A, B, C and D strains of C. neoformans, such epitopes containing acetyl groups in the polysaccharide. Other monoclonal antibodies of this invention, which are specific for serotype D strain C. neoformans only, recognize protective epitopes on the polysaccharide of serotype D strain C. neoformans only. This invention is further directed to methods for producing these monoclonal antibodies.

The monoclonal antibodies of this invention are produced by infecting animals with the C. neoformans organism, or by immunizing animals with a glycoconjugate comprised of CNPS conjugated to a protein carrier, performing an ELISA to determine which animals have high serum titers of antibody to the C. neoformans and fusing the spleen cells of high serum titer animals with NSO myeloma cells to produce hybridomas which secrete monoclonal antibodies. These monoclonal antibodies may be used to treat cryptococcal infection, such as Cryptococcal meningitis, especially in immunosuppressed patients, and may also be used to prevent cryptococcal infection by passive administration. In addition, these monoclonal antibodies react with Trichosporon antigens and may be used to treat Trichosporon infections. The monoclonal antibodies of this invention may be used for detection of fungal infection, diagnostic serotyping and anti-fungal therapy. These monoclonal antibodies may also be used to diminish the level of C. neoformans polysaccharide circulating in body fluids.

### BRIEF DESCRIPTION OF THE DRAWINGS

FIG. 1 represents serum antibody responses of three responder Balb/c mice infected with the GH cryptococcal strain. The bars represent the antibody titer in terms of IgM, total IgG, κ, and λ at various time points after infection. Antibody titer was measured by serial dilutions on ELISA plates coated with 10 μg/ml of GH CNPS. The titer was defined as the serum dilution which gave an optical density at 405 nm which was at least 1.5 times the background in the ELISA. The three mice (aged 9-12 months) were infected with sublethal innocula of 10⁴-10⁶ cryptococci intraperitoneally. The IgG fraction of mouse 3 consisted exclusively of IgG₁ and IgG₃ subtypes.

FIG. 2 represents ELISA binding data of the 14A12 and 21D2 monoclonal antibodies to GH CNPS. The graph shows a plot at OD₄₀₅ nm versus GH CNPS concentration where the monoclonal antibody concentration is kept constant at 1 μg/ml and the CNPS concentration is varied. The binding curves of the other IgMλ0 antibodies, 7B13, llE2, 12G5, 20B5, and 20C5 were like that of the 14A12 monoclonal antibody and are not shown here. The hybridoma supernatants were screened using plates coated with 10 μg/ml of GH CNPS.

FIG. 3 represents serum antibody titer of the two mice which manifested a rise in serum anti-CNPS titer following intraperitoneally infection with 10⁵ serotype A cryptococci. Open bars are IgM and closed bars are IgG. Seven monoclonal antibodies were generated from the spleen of mouse Al but none were obtained from the spleen of mouse A2. Infection with this innocula of cryptococci was not lethal after five months of observation.

FIG. 4 represents serum antibody titer of six mice immunized with serotype A CNPS-tetanus toxoid glycoconjugate. Open bars are IgM and closed bars are IgG. Mice B1-B4 received 10 intraperitoneal injections of 2.5 μg conjugate in PBS on day 1. Mouse B4 was given a third injection of 2.5 μg conjugate on day 37 and the spleen was harvested for fusion on day 40. Mice F1 and F2 were given a single injection of 2.5 μg of conjugate intraperitoneally.

FIG. 5 represents the protective efficacy of the monoclonal antibodies of this invention. Protective efficacy is demonstrated by the ability of these monoclonal antibodies to prolong survival in lethally infected animals. The data shows that the protective efficacy of the different isotypes is IgG₁>IgM>IgA>IgG₃. The 2H1 monoclonal antibody of this invention completely protected 50% of the lethally infected mice for more than 100 days.

FIG. 6 represents the binding of monoclonal antibodies of this invention to native and de-O-acetylated GXMs from two serotype A strains. Panels A-C and D-F show this binding to GXM of ATCC strain 24064 and isolate 371, respectively. Monoclonal antibodies 5E9 (IgM_{κ}) and 3B10 (IgG_{κ}) were generated from a mouse infected with strain ATCC 24064. Monoclonal antibodies 13F1 (IgM_{κ}), 2H1 (IgG_{κ}), and 18G9 (IgA_{κ}) were generated from a conjugate-immunized mouse. Monoclonal antibody 21D2 (IgM_{κ}) was generated from a mouse infected with the clinical isolate GH. The 21D2 monoclonal antibody behaves like 5E9 in its reactivity with native and de-O-acetylated serotype A GXM.

Table I represents class, light chain usage, and reactivity with CNPS of serotypes A, B, C, D, and GH, and the V_{H} and V_{L} usage for the anti-CNPS monoclonal antibodies. The symbols “+” and “−” denote binding and lack of binding respectively to ELISA plates coated with 10 μg/ml of CNPS from the different serotypes. The 15C6 monoclonal antibody is separated from the others by a dashed line because it was generated from the spleen of a different mouse. The V_{H}, J_{H}, V_{L}, and J_{L} were determined from the Ig mRNA sequences. The V_{H} Ga150.1 and V_{H}441 are gene elements belonging to the 7183 and X-24 gene families respectively.

Table II represents isotype, light chain usage, and reactivity with CNPS of serotypes A, B, C, and D as well as that of strain GH for the various monoclonal antibodies obtained from mice infected with the actual organism and with the glycoconjugate.

### DETAILED DESCRIPTION OF THE INVENTION

C. neoformans is an opportunistic fungal infection which is dangerous and often fatal in immunosuppressed patients. The monoclonal antibodies produced by the present invention may be administered passively to aid in the treatment and prevention of cryptococcal infection, such as Cryptococcal meningitis. In addition, they may be used to treat Trichosporon infections. They may also be used for the detection of fungal infection, the development of diagnostic serotyping of clinical isolates, and as therapeutic adjuncts to anti-fungal antibiotic therapy. Further, the antibodies of this invention may be used to reduce the level of C. neoformans polysaccharide circulating in body fluids.

Some of the monoclonal antibodies of this invention bind to all four serotype A, B, C and D strains of the C. neoformans fungus. These monoclonal antibodies bind to epitopes on C. neoformans which are non-enhancing protective epitopes, such epitopes containing acetyl groups in the polysaccharide. These monoclonal antibodies are more effective at conferring protection against all cryptococcal infections. Other monoclonal antibodies of this invention are serotype specific, and bind to protective epitopes on the polysaccharide capsule of serotype D strain C. neoformans only.

The method of producing the monoclonal antibodies of the present invention comprises infecting animals with either the C. neoformans organism itself, or immunizing animals with a conjugate of the capsular polysaccharide of C. neoformans (CNPS) and a protein carrier, such as tetanus toxoid to form a glycoconjugate. After either infection with the C. neoformans organism or immunization with the glycoconjugate, an ELISA is performed to determine the presence of antibody to C. neoformans in the sera of the animals. Those animals with high serum titers of antibody to C. neoformans are used for the production of monoclonal antibodies. The spleen cells of those animals and NSO myeloma cells are fused to produce hybridomas which secrete monoclonal antibodies. After production of the monoclonal antibodies, the monoclonal antibodies are screened by ELISA, cloned in soft agar and administered passively to animals.

The monoclonal antibodies of this invention which are specific for serotype A, B, C and D strains of C. neoformans were generated from hybridomas produced by the fusion of NSO myeloma cells with splenocytes of animals either immunized with the CNPS-tetanus toxoid conjugate or infected with the C. neoformans organism. The monoclonal antibodies which are specific for serotype D strain only of C. neoformans were generated from hybridomas produced by the fusion of NSO myeloma cells with splenocytes of animals infected with the GH strain C. neoformans organism.

### EXAMPLE 1

### Isolation of C. neoformans

C. neoformans was used to infect mice. The strain was isolated from the cerebrospinal fluid of an AIDS patient with cryptococcal meningitis, and we denoted this strain “GH.” Standard serotype strains, A, B, C, and D (ATCC numbers 24064, 24065, 24066, and 24067 respectively) were obtained from the American Type Culture Collection, Maryland. C. neoformans capsule polysaccharide (CNPS) was prepared as described by Kozel, T. R., and Cazin, R., “Nonencapsulated Variant of Cryptococcus neoformans”, Infect. Immuno. 3: 287-294 (1971) and Dromer, F., Salamero, J., Contrepois, A., Carbon, C., and Yeni, P., “Production, Characterization and Antibody Specificity of a Mouse Monoclonal Antibody Reactive with Cryptococcus neoformans Capsular Polysaccharide”, Infect. Immun. 55: 742-748 (1987). The concentration of polysaccharide was determined by the phenol-sulfuric acid method. See Dubois, M., Gilles, R. A., Hamilton, J. K., Rebens, P. A., and Smith,. F., “Colorimetric Method for Determination of Sugars and Related Substances”, Anal. Chem. 28: 350-356 (1956). Yeast used in the isolation were maintained in Sabouraud's agar slants at 4° C.

### Infection of Mice

Balb/c mice were obtained from the National Cancer Institute. The mice were infected with the A strain of actual cryptococcus organism intraperitoneally. Prior to innoculation, the yeast were washed with PBS and counted in a hemocytometer. After infection, the mice were bled from the retro-orbital sinus, and sera were separated by centrifugation and stored at −20° C.

### Titer Analysis of Infected Mice by ELISA

To perform an ELISA so that titer of antibody in the infected mouse sera could be determined, Corning ELISA Plates (No. 25801) were coated with CNPS by incubating 50 μl of a 10 μg/ml solution of CNPS in 0.02 M phosphate buffered saline, ph 7.2, (PBS) in each well at room temperature overnight. Plates were blocked with a solution of 1% bovine serum albumin (BSA) in PBS. Fisher Biotech alkaline phosphatase conjugated goat anti-mouse IgM, IgG₁, IgG₃, IgG₂ₐ, IgG_{2b}, IgA, κ, and λ reagents were used to develop the ELISA.

### Generation of Monoclonal Antibodies

Monoclonal antibodies to C. neoformans CNPS were made from chronically infected Balb/c mice with high serum titers. The use of spleens from infected mice posed the potential problem of hybridoma cell culture contamination with cryptococci. This problem was avoided by treating the high serum titer mice with Amphotericin B and the hybridoma cultures with Nystatin. The mice were treated with Amphotericin B intraperitoneally (5-15 mg/kg total dose) during the week prior to harvesting the spleens to decrease the number of cryptococci in their tissues. The brain, heart, lungs, liver, and kidney from a mouse that had received 15 mg/kg of Amphotericin B were cultured, and cryptococci was found only in brain tissue. The generation of monoclonal antibodies from infected mice has not been done by others, possibly because of the high likelihood of the contamination of tissues by fungus. Amphotericin B was administered so that this problem was avoided.

Hybridomas were made by fusing splenocytes from the high serum titer mice with NSO myeloma cells at a 4:1 ratio with polyethylene glycol by a protocol described in Fazekas, S., Groth, S. T., and Scheidagger, D., “Production of Monoclonal Antibodies: Strategy and Tactics”, J. Immunol. Methods 35: 1-21 (1980). Nystatin (Gibco) was added to the hybridoma cultures at a concentration of 100 units/ml one day after the fusion. Hybridomas were then screened by ELISA using plates coated with 50 μl of 10 μg/ml GH CNPS. Cells from positive wells were cloned in soft agar. For the selection of some anti-CNPS monoclonal antibody hybridomas, soft agar plates were overlaid with agar containing 10-50 μg/ml of CNPS. This resulted in a faint antigen-antibody precipitate around anti-CNPS producing colonies which aided in their selection.

### Isotype and ELISA Chain Analysis

The monoclonal antibody isotypes and light chain types were determined using goat anti-mouse isotype and light chain specific alkaline phosphatase labelled antibodies. Hybridoma supernatants containing the monoclonal antibodies were used for binding studies. The monoclonal antibody concentration for all hybridomas was determined by ELISA relative to standards of the same isotype and of known concentration. Because the goat anti-IgG₃ reagents were of low affinity, 4H3 monoclonal antibodies were purified using an anti-mouse IgG column, dialyzed against PBS, and their concentration was determined by the Bio-Rad protein assay using a myeloma IgG₃ as a standard rather than by ELISA.

### Results

Sixty Balb/c mice were infected with GH strain C. neoformans. Only four out of the sixty mice had a detectable increase in serum anti-CNPS. Upon analysis, the sera of three responder mice contained both IgM and IgG anti-CNPS antibodies, and the titer of the λ and κ anti-CNPS antibody were approximately equal. 7 IgM and 1 IgG₃ monoclonal antibodies were generated from the spleen of one responder mouse, and 1 IgA was generated from the spleen of another mouse.

Seven of the IgM's, the IgG₃, and the IgA monoclonal antibodies had λ light chains and were specific for serotype D strain CNPS only. All of these monoclonal antibodies contained V_{H}441, J_{H}3 and either V_{λ2}/J_{λ2} or V_{λ1}1/J_{λ1}, and all had the same heavy chain CDR3 amino acid sequence even though there were differences in the nucleotide sequence of the N/D segment. Southern blot analysis of J locus rearrangement of the heavy and light alleles indicated that the serotype D strain CNPS specific monoclonal antibodies arose from only a few precursor B cells. One IgM monoclonal antibody reacted with serotype A, B, C and D strains CNPS. This monoclonal antibody utilized different V_{H} and J_{H} genetic elements, and had κ light chains. All of the anti-CNPS monoclonal antibodies utilized J proximal V_{H} gene elements that had previously been shown to bind dextran and other polysaccharides.

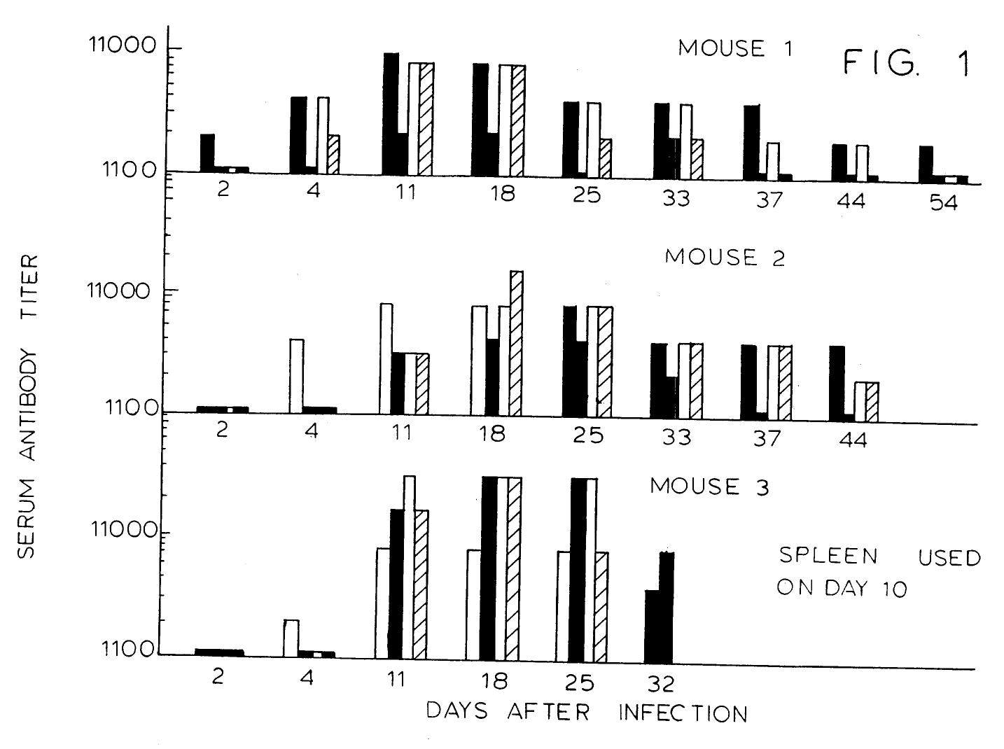

FIG. 1 shows the serum titers of IgM, IgG, κ and λ at several times after injection for three of the mice which had high titers of anti-CNPS. This data shows that the antibody titers peaked at about 11-18 days and then slowly declined with time, even though these animals were chronically infected. This data also shows that both IgM and IgG are present. Finally, this data shows that in many of the bleedings, the titer of λ is roughly equivalent to that of κ. The two spleens from the mice with the highest titers of antibodies to CNPS were used. One spleen yielded 7 IgM and 1 IgG₃ monoclonal antibodies. The other spleen yielded only 1 IgA monoclonal antibody.

The monoclonal antibodies were characterized for heavy chain iso,type, light chain type and binding to CNPS from the standard ATCC A, B, C, and D serotypes and the GH strain (see Table I below). Although the serotype of the GH strain used in this study was not initially known, the reactivity of the monoclonal antibodies with GH CNPS suggests that GH belongs to the D serotype. The monoclonal antibodies were named 21D2, 14A12, 4H3 and 15C6. The 14A12 (μλ) group of antibodies, which are IgM λ antibodies, includes antibodies 7B13, 11E2, 12G5, 20B5 and 20C5. The 14A12 group of IgM antibodies are specific for only serotype D strain CNPS. Monoclonal antibody 21D2 (μκ) is also an IgM κ antibody, which binds to serotype A, B, C and D strains CNPS. Monoclonal antibody 4H3 is an IgG₃λ antibody, and is specific for only serotype D strain CNPS. Finally, monoclonal antibody 15C6 (αλ) is an IgA λ antibody, and is specific for only serotype D strain CNPS.

TABLE I
CHARACTERISTICS OF CNPS BINDING ANTIBODIES
Serotype Polysaccharide

| Mono- | | | | | | | | | | |
|---|---|---|---|---|---|---|---|---|---|---|
| clonal | Class | A | B | C | D | GH | V_{H} | J_{H} | V_{L} | J_{L} |
| 21D2 | IgMκ | + | + | + | + | + | 7183- | 2 | V_{κ} | J_{κ}1 |
| | | | | | | | 283 | | 5.1 | |
| 14A12 | IgMλ | − | − | − | + | + | V_{H}441 | 3 | V_{λ}2 | J_{λ}2 |
| 11E2 | IgMλ | − | − | − | + | + | V_{H}441 | 3 | V_{λ}2 | J_{λ}2 |
| 7B13 | IgMλ | − | − | − | + | + | V_{H}441 | 3 | V_{λ}2 | J_{λ}2 |
| 12G5 | IgMλ | − | − | − | + | + | V_{H}441 | 3 | V_{λ}2 | J_{λ}2 |
| 20C5 | IgMλ | − | − | − | + | + | V_{H}441 | 3 | V_{λ}2 | J_{λ}2 |
| 20B5 | IgMλ | − | − | − | + | + | V_{H}441 | 3 | V_{λ}2 | J_{λ}2 |
| 4H3 | IgG3λ | − | − | − | + | + | V_{H}441 | 3 | V_{λ}1 | J_{λ}1 |
| 15C6 | IgAλ | − | − | − | + | + | V_{H}441 | 3 | V_{λ}2 | J_{λ}2 |

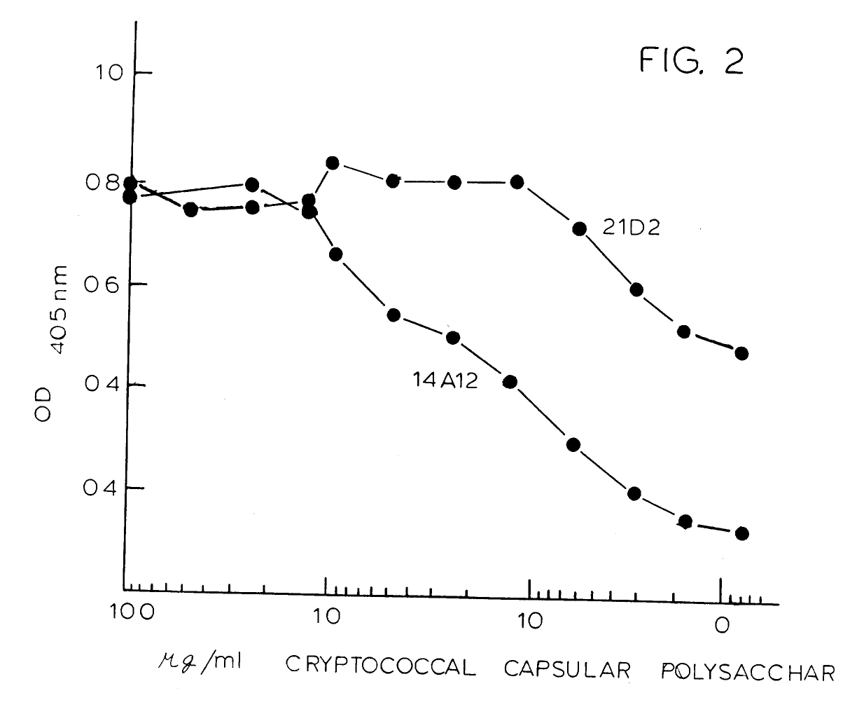

FIG. 2 shows the binding curves of 14A12 (μλ) and 21D2 (μκ) to GH CNPS. The binding curves of the other IgL λ antibodies, 713, 11E2, 12G5, 20B5 and 20C5 are indistinguishable from those of the 14A12, and are not shown in FIG. 2. The binding curves of 14A12 and 21D2 are not directly comparable since the two antibodies bind to different epitopes.

All of the serotype D strain specific λ monoclonal antibodies have a heavy chain variable region (V_{H}) encoded by V_{H} 441, a small “diversity” segment consisting of four codons and J_{H}3. The light chain variable region (V_{L}) is encoded by Vλ2/Jλ2 for the 14A12 class and 15C6, and by Vλ1/Jλ1 for 4H3. The fact that all of these monoclonal antibodies have a variable region structure which is identical or nearly identical indicates that they all recognize the same epitopes.

The construction of the 21D2 monoclonal antibody was different than that of the serotype D strain specific monoclonal antibodies. 21D2, which is specific for serotype A, B, C and D strains, is composed of V_{H}7183-283, an unidentified diversity segment, and J_{H}2. The diversity segment of 21D2 has seven codons and is thus larger than that found in the serotype D specific monoclonal antibodies. The light chain of 21D2 is composed of V_{κ}5.1 and J_{κ}2.

### EXAMPLE 2

### Isolation of C. neoformans

Balb/c mice were obtained from the National Cancer Institute. Cryptococcal strains of serotypes A, B, C, and D were obtained from the American Type Culture Collection (ATCC numbers 24064, 24065, 24066 and 24067 respectively). Capsular polysaccharide was prepared as described by others. See Kozel, T. R., and Cazin, R., “Nonencapsulated Variant of Cryptococcus neoformans”, Infect. Immuno. 3: 287-294, 1971.

### Preparation of Glycoconjugates

The glycoconjugates were prepared as described in Devi et al., “Glucuronoxylomannan-Protein Conjugate Vaccines of Cryptococcus Neoformans, Serotype A: Synthesis, Characterization and Immunogenicity”, Infect. Immun. 59: 3700-3707 (October, 1991). The glucuronoxylomannan (GXM) of serotype A strain C. neoformans was purified by precipitation with cetyltrimethylammonium bromide (CTBA). The capsular polysaccharide of serotype A strain C. neoformans was dissolved in 0.2M NaCl and was then mixed with 10% CTBA to a final concentration of 0.39% with constant stirring at room temperature. The precipitate was collected by centrifugation at 16,000 g for 1 hour and the supernatant was reprecipitated with 0.05% cetavlon. The precipitates were dissociated in 1M NaCl and deproteinized by cold phenol extraction, dialysed extensively against sterile pyrogen-free water and freeze dried. This material was denoted as native GXM.

The native GXM was depolymerized by ultrasonic irradiation (Heat System Ultrasonicator, model w225R) at a power setting of 2 and pulse of 90% for 1.5 hours in an ice bath. The sonicated GXM was subject to gel filtration through Sepharose 2B-CL column (1.5×30 cm). The GXM-containing fractions eluting at about the middle of the column were collected, dialysed against pyrogen-free water at 3-8° C., sterile filtered (0.45 μm) and freeze-dried. This sonicated material was assigned the general term GXM.

ADH was introduced into GXM by activation of hydroxyl groups with CNBr. GXM (5 mg/ml of 0.2M NaCl) was activated with an equal weight of CNBr at pH 10.5 for 6 minutes at 4° C. using a pH Stat. An equal volume of 0.5M ADH dissolved in 0.5M NaHCO₃, pH 8.5 was added. The reaction mixture was tumbled at 3-8° C. for 18-20 hours, dialyzed against 0.2M NaCl and passed through 2B-CL Sepharose column (1.5×30 cm). The fractions containing GXM were pooled and concentrated to the original volume.

The reaction mixture containing equal concentrations (3.0 to 7.5 mg/ml) of GXM-AH and tetanus toxoid (TT) in 0.2M NaCl was brought to pH 5.6 with 0.05N HC1, and 0.05-0.1M EDAC was added. The pH was maintained at 5.6 in a pH Stat for 1-3 hours at 4° C. The reaction mixture was dialysed against 0.2M NaCl at 3-8° C. and passed through Sepharose 2B-CL column (1.5×30 cm) equilibrated in 0.2M NaCl. The void volume fractions containing the GXM and the protein were pooled and stored in 0.01% thimerosal at 3-8° C. The conjugate GMX-TT was prepared through hydroxyl activation.

### Infection of Mice

Cryptococci (serotype A, strain ATCC 24064) were washed and resuspended in phosphate buffered saline, pH 7.2 (PBS). Each mouse was infected with 10⁵ cryptococci intraperitoneally. Innocula was determined by counting the yeast in a hemocytometer and confirmed by plating on Sabouraud's agar. Other mice were immunized with the glycoconjugate intraperitoneally with and without Freund's complete adjuvant. All mice were bled from the retro-orbital sinus and sera were stored at −20° C.

### Titer Analysis of Infected Mice by ELISA

Serum antibody titers were measured by ELISA. The titer was defined as the greatest dilution which gave an optical density of 1.5 times the background. The ELISA used Corning plates (No. 25801) coated with a solution of 10 μg/ml of CNPS in 0.020 M phosphate buffered saline (PBS), and blocked with a solution of 1% bovine serum albumin (BSA) and 0.5% horse serum in PBS.

### Generation of Monoclonal Antibodies

Monoclonal antibodies were generated from a mouse infected with the serotype A strain C. neoformans organism and from a mouse immunized with the glycoconjugate in saline. Infected animals were treated with Amphotericin B (15 mg/kg intraperitoneally) during the week prior to the fusion to decrease the possibility of cryptococcal contamination of hybridoma tissue cultures. In addition, Nystatin was added to the hybridoma cultures at a concentration of 100 units/ml 24 hours after fusing the splenocytes and NSO myeloma cells. No cryptococcal contamination of the tissue cultures was observed.

Hybridomas were made by fusing splenocytes with NSO myeloma cells at a ratio of 4:1 using polyethylene glycol as described by Fazekas et al. See Fazekas, S., Rowth, S. T., and Scheidagger, D., “Production of Monoclonal Antibodies: Strategy and Tactics”, J. Immunol Methods 35, 1-21 (1980). Each hybridoma supernatant was screened by ELISA simultaneously on plates coated with serotype A CNPS and GH CNPS.

The primary screen was performed using GH CNPS instead of the CNPS from the ATCC D serotype because the immune sera produced stronger signals with GH. Blocking solution was used to eliminate BSA and plate binding monoclonal antibodies. ELISAs were developed with a mixture of FisherBiotech alkaline phosphatase labelled goat anti-mouse IgM, IgG₁, IgG₂ₐ, IgG_{2b}, IgG₃, and IgA. Isotype was determined using these same reagents and light chain type was determined using alkaline phosphatase goat anti-mouse λ and κ (FisherBiotech). Hybridomas producing anti-CNPS monoclonal antibodies were cloned twice in soft agar.

### Results

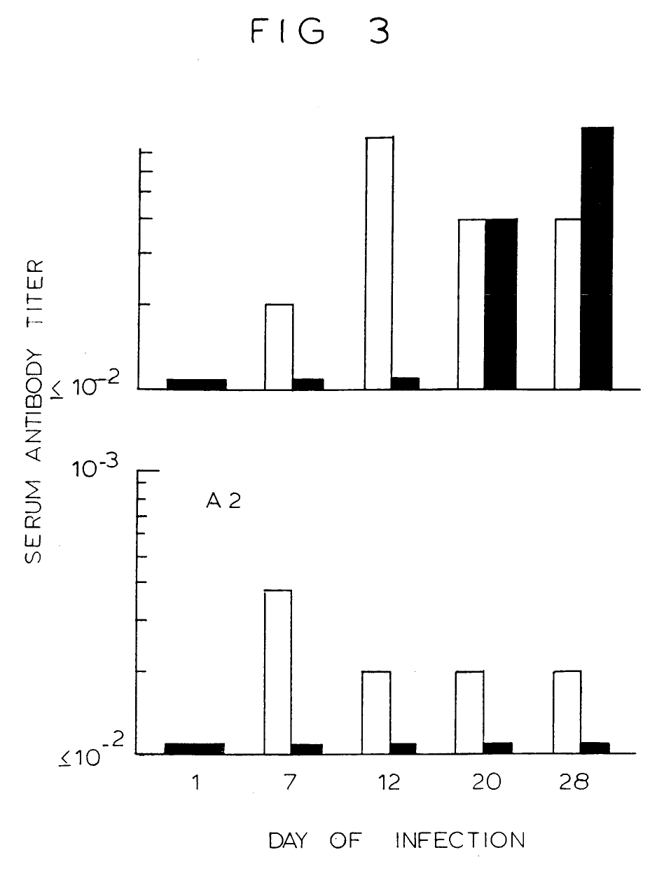

Infection of Balb/c mice with the serotype A cryptococci organism elicited a rise in anti-CNPS titer in only two out of 24 mice. The serum titers of anti-CNPS IgM and IgG at several times after infection are shown in FIG. 3. Animal Al made both IgM and IgG. The IgG component of the titer was predominantly IgG₁. The serum antibody response of Animal A2 was limited to a small increase in IgM anti-CNPS titer.

Spleen cells from both the A1 and A2 mice were fused to NSO myeloma cells. Anti-CNPS hybridomas were obtained only from mouse Al, suggesting that the A2 spleen contained fewer antibody-producing cells.

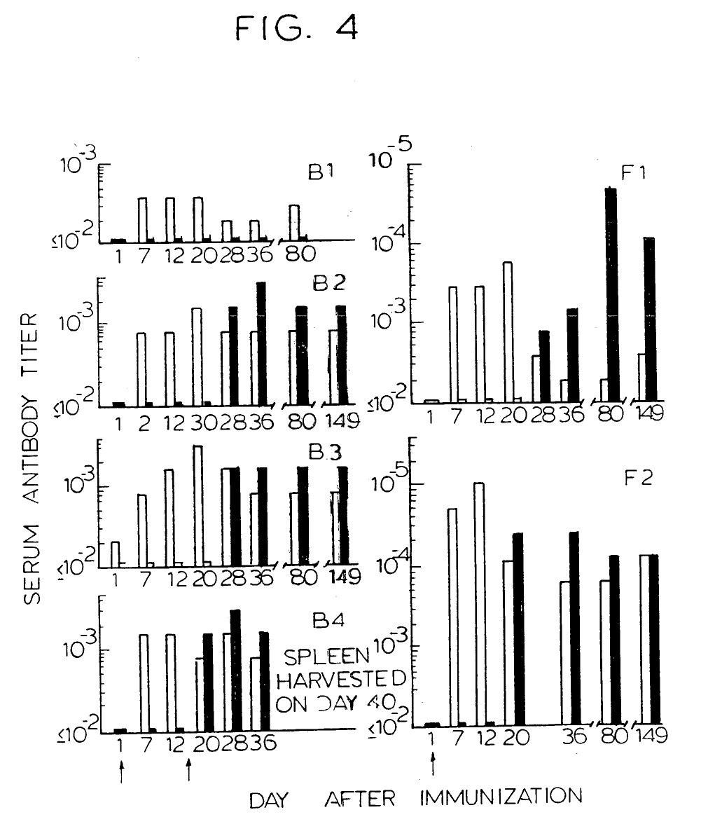

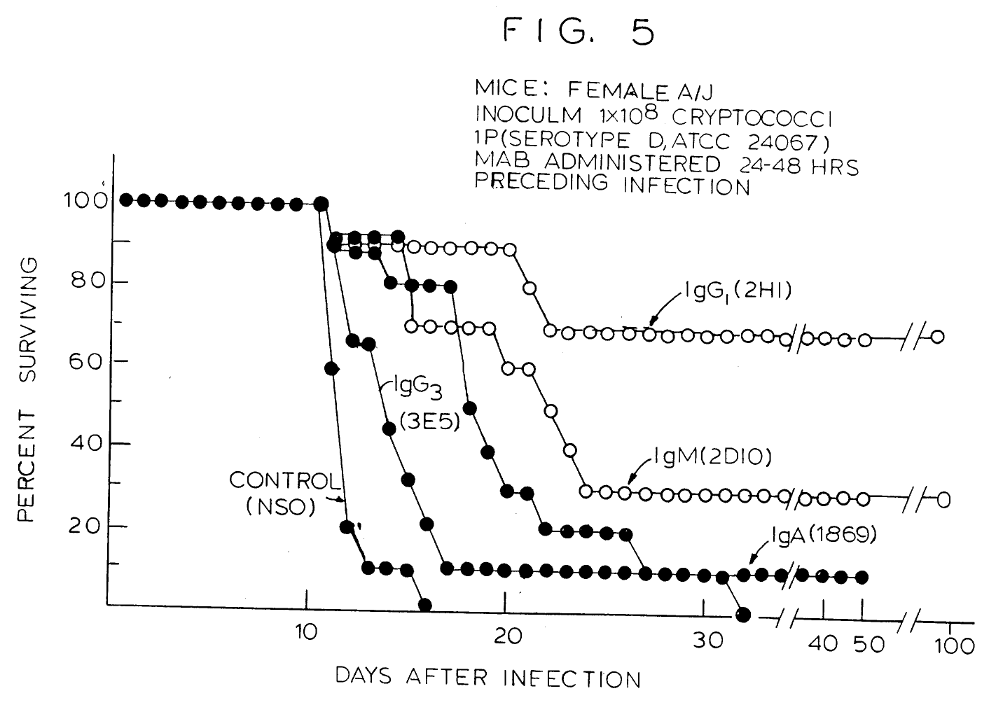

Six mice were immunized with serotype A CNPS-tetanus toxoid glycoconjugate. All six mice produced an increase in serum anti-CNPS titer. FIG. 4 shows the IgM and IgG serum titers of four mice given two injections of glycoconjugate intraperitoneally, and two mice given glycoconjugate in CFA intraperitoneally. Three of the four animals given two dosages of glycoconjugate at days 1 and 14 made serum IgG after the second dose. The IgG was predominantly IgG₁. Mouse B4 was then given a third dose of glycoconjugate intraperitoneally on day 37, and the spleen cells were fused to NSO myeloma cells on day 40, resulting in over 30 anti-CNPS hybridomas. Immunization of two mice with a single dose of glycoconjugate in CFA resulted in high titers of both anti-CNPS IgM and IgG. (See FIG. 4). The hybridomas which generate the monoclonal antibodies of this invention may be altered by sib selection so that they express different isotype classes and subclasses in order to make the antibodies more useful. See Aguila, H., French, D., and Scharff, M., “Class and Subclass Switching of Hybridomas In Vitro”, Immunochemica, Vol. 2, No. 2, 1-4 (June 1988).

Both cryptococcal infection with the serotype A 10 organism and immunization with the glycoconjugate induced the production of antibodies that reacted with other serotypes. The IgM and IgG titers to serotype D strain CNPS were comparable to those observed for serotype A CNPS. In contrast, the serum responses to CNPS of serotypes B and C were weaker and consisted of only an increase in IgM. This is consistent with the fact that the various serotypes are known to share epitopes, and that A and D serotypes are related antigenically.

Seven monoclonal antibodies were made from the spleen cells of mouse Al, which was infected with the serotype A strain organism, and 31 monoclonal antibodies were made from the spleen cells of mouse B4, which was immunized with glycoconjugate. No anti-CNPS hybridomas were generated from the infected A2 mouse, which had only a low titer of IgM. The class, subclass and CNPS serotype specificity of the monoclonal antibodies are shown in Table II.

TABLE II
Mabs generated from infected and conjugate Immunized Mice.

| | | | CNPS | | SEROTYPE | | |
|---|---|---|---|---|---|---|---|
| ANIMAL | CLASS | NUMBER | A | B | C | D | GH |
| A1 (infected) | IGM_{κ} | 6 | + | + | + | + | + |
| | IgG₁κ | 1 | + | + | + | + | + |
| B4 (conjugate) | IgM_{κ} | 9 | + | + | + | + | + |
| | IgG₃κ | 1 | + | + | + | + | + |
| | IgG₁κ | 16 | + | + | + | + | + |
| | IgAκ | 7 | + | + | + | + | + |

Of the seven monoclonal antibodies obtained from mouse A1, six were IgM and one was IgG . This is consistent with the isotype distributions expected in the antibody response to a presumed T-independent antigen such as CNPS. In contrast, the glycoconjugate immunized mouse (B4) yielded 31 monoclonal antibodies, of which nine were IgM, one was IgG₃, 16 were IgG₁ and seven were IgA. The predominance of the IgG class in the monoclonal antibodies from mouse B4 is consistent with and strongly suggests a T-dependent response. The monoclonal antibodies obtained from the mouse immunized with the glycoconjugate had K light chains, and were composed of V_{H}7183-283, seven amino acid diversity segments, J_{H}2, V_{κ}5.1 and J_{κ}1. The sera of the six conjugate immunized mice was analyzed at day 36, and anti-CNPS IgA was found in the sera of three mice.

Some of the monoclonal antibodies generated in response to infection with the C. neoformans organism were specific for serotype D strain only. Other monoclonal antibodies generated in response to the infection with the C. neoformans organism and all of the monoclonal antibodies generated in response to the glycoconjugate immunization were specific for serotype A, B, C and D strains of C. neoformans.

The serum antibody responses to CNPS induced by infection with the C. neoformans organism and immunization with the glycoconjugate were of the same class, subclass and specificity. (See Table II.) This indicates that the same antigenic determinant is presented to the mice by infecting with the A strain of C. neoformans and by infecting with the glycoconjugate. The conjugation of CNPS to tetanus toxoid presents this determinant and enhances its immunogenicity. Further, the glycoconjugate immunization resulted in monoclonal antibodies which were specific for all strains of cryptococci, namely serotype A, B, C and D strains. Hence, the serotype A, B, C and D strain-specific monoclonal antibodies of this invention may be used to treat and prevent infection from all strains of cryptococcal fungus.

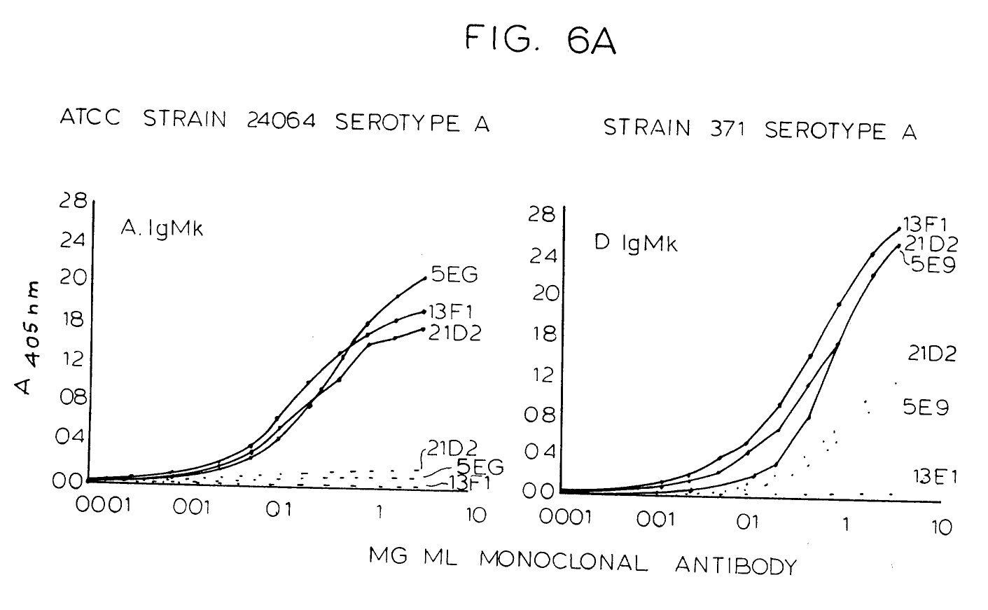

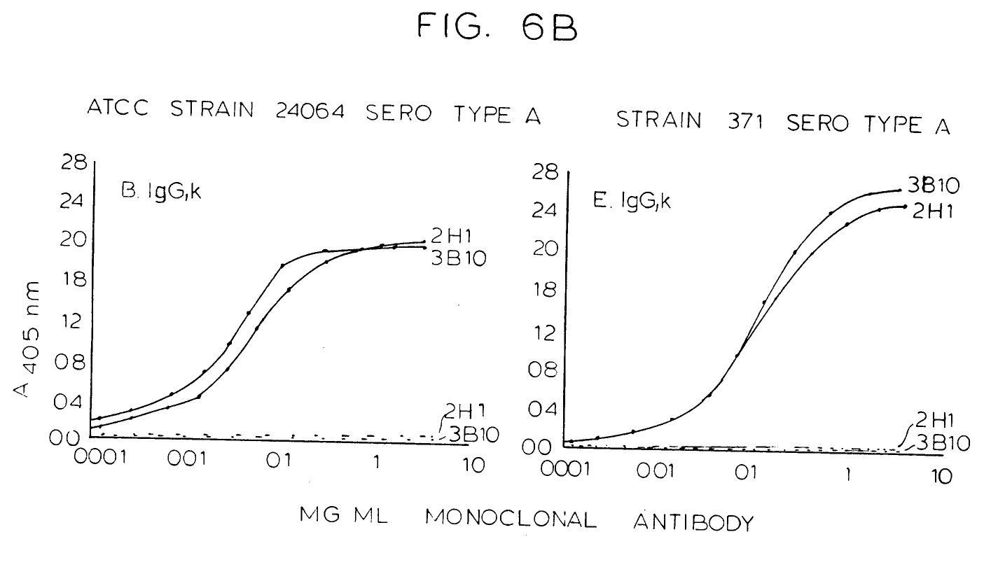

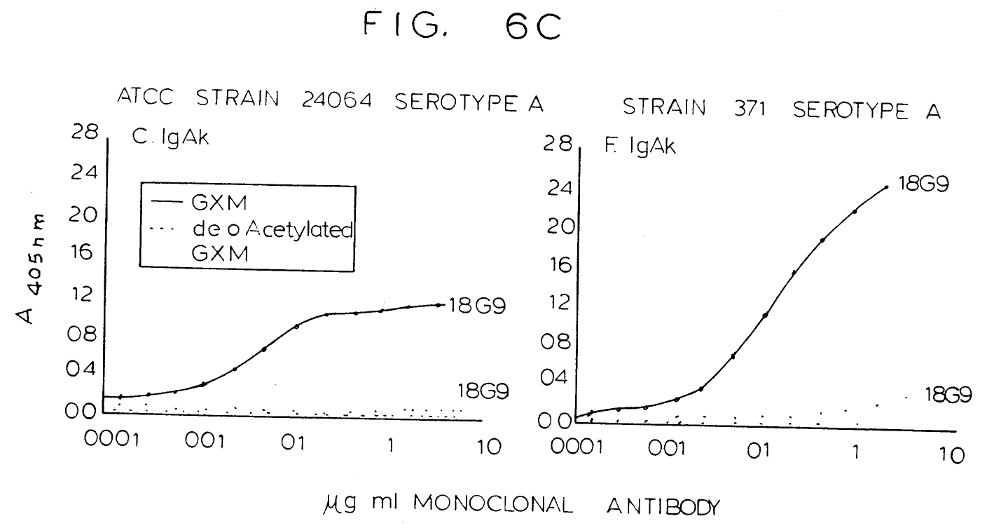

In order to determine which epitopes were recognized by the monoclonal antibodies of this invention, we studied the binding of the monoclonal antibodies to GXM which had been modified by removal of acetyl groups. FIG. 6 shows the binding data of the monoclonal antibodies to the GXM from two different serotype A strains. The antibodies shown are 5E9 (IgM) and 3B10 (IgG₁) (generated from the infected mouse); 13F1 (IgM), 2H1 (IgG₁) and 18G9 (IgA) (which were generated from the conjugate-immunized mouse); and 21D2 (IgM). A deposit of the 2H1 antibody-producing hybridoma was made with the American Type Culture Collection on Oct. 14, 1991 and catalogued as ATCC #HB 10902. For the GXM of strain 371, de-O-acetylation abolished the binding of monoclonal antibodies of 3B10, 13F1, 2H1 and 18G9. However, de-O-acetylation only reduced the binding of 5E9 and 21D2. This indicates that O-acetyl groups are an important portion of the epitope recognized in the GXM. The residual binding of monoclonal antibodies 5E9 and 21D2 to strain 371 de-O-acetylated GXM may reflect polysaccharide structural differences between strains 371 and 24064, or different epitope specificities between the monoclonal antibodies.

### Antibody Protection Studies

The ability of the monoclonal antibodies of this invention to confer protection was determined in a mouse model of cryptococcal infection. Preventive efficacy was measured as the capacity of a monoclonal antibody to prolong survival in lethally infected mice. This preventive efficacy was measured in comparison to an untreated control group of mice. The control mice were infected intraperitoneally with a dose of 10⁸ cryptococci per mouse. The cryptococcal strain was obtained from the American Type Culture Collection, ATCC No. 24067. The control group received an irrelevant antibody, irrelevant ascites fluid (NSO myeloma ascites) or phosphate buffered saline. In the experimental group, the monoclonal antibody was administered shortly before cryptococcal innoculation. The mice were observed daily, and there was a reduction in the amount of cryptococcal polysaccharide in the serum of the treated animals, in comparison to the level of cryptococcal polysaccharide in the serum of the control group. The monoclonal antibodies of this invention were able to significantly prolong the survival of lethally infected mice, and also resulted in a reduction of serum polysaccharide concentrations.

It is possible to develop chimeric mouse-human antibodies using murine antibodies as developed by the methods of this invention. Chimeric mouse-human antibodies contain variable regions from murine hybridomas and human constant regions. To produce chimeric mouse-human antibodies, mouse variable regions specific for a given antigen are obtained from a hybridoma and then joined by recombinant DNA techniques to human constant regions, which are usually obtained from genomic clones. The resulting chimeric genes are then transfected into a recipient cell line, and transfectoma cell lines synthesizing functional antibodies are identified and isolated for in vivo or in vitro amplification. See Morrison, S., “Genetically Engineered (Chimeric) Antibodies”, Hospital Practice, 65-80 (Oct. 15, 1989).

The sequence data for the antigen-binding portion of a monoclonal antibody specific for serotype A, B, C and D strains of C. neoformans which has non-enhancing protective epitopes containing acetyl groups, determined by the methods outlined herein, is as follows:

| | | |
|---|---|---|
| | 1 10 | |
| V_{H}7183-283 | GAA GTG ATG CTG GTG GAG TCT GGG GGA GGC TTA GTG AAG CCT | SEQ. ID. NO.1 |
| 4D4 | --C --- -AT --C --- --- --- --- --- --- --- --- --- -T- | |
| | 20 | |
| | GGA GGG TCC CTG AAA CTC TCC TGT GCA GCC TCT GGA TTC ACT | |
| 4D4 | --- --- --- --- --- --- --- --- --- --- --- --- --- --- | |
| | 30 40 | |
| | TTC AGT AGC TAT ACC ATG TCT TGG GTT CGC CAG ACT COG GAG | |
| 4D4 | --- --- --- --- TT- --- --- --- --- --- --- --- --A --- | |
| | 50 | |
| | AAG AGG CTG GAG TGG GTC GCA ACC ATT AGT AGT GGT GGT GGT | |
| 4D4 | --- --- --- --- -T- --- --- -TG --- -A- -A- -A- --- TT- | |
| | 60 70 | |
| | AAC ACC TAC TAT CCA GAC AGT GTG AAG GGT CGA TTC ACC ATC | |
| 4D4 | --- --- --- --- --- --- -C- --- --- --G --- --- --- --- | |
| | 80 | |
| | TCC AGA GAC AAT GCC AAG AAC AAC CTG TAC CTG CAA ATG AGC | |
| 4D4 | --- --- --- --- --- --- --- -C- --- --- --- --- --- --- | |
| | 90 | |
| | AGT CTG AGG TCT GAG GAC ACG GCC TTG TAT TAC TGT GCA AGA | |
| 4D4 | --- --- -A- --- --- --- --A --- --- --- --- --- --- --- | |
| D segment | | |
| 4D4 | CGT GAT GCT TAC TTT TCG CAC | SEQ. ID. NO.2 |
| J_{H}2 | TAC TTT GAC TAC TGG GGC CAA GGC ACC ACT CTC ACA GTC | SEQ. ID. NO.3 |
| 4D4 | --- --- --- --- --- --- --- --- --- --- --- --- --- | |
| | TCC TCA | |
| 4D4 | --- --- | |
| | 1 10 | |
| V_{κ}5.1 | AGT GAT GTT GTG ATG ACC CAA ACT CCA CTC TCC CTG CCT GTC | SEQ. ID. NO.4 |
| 4D4 | --- --- --- --- --- --- --- --- --- --- --- --- --- -A- | |
| | 20 | |
| | AGT CTT GGA GAT CAA GCC TCC ATC TCT TGC AGA TCT AGT CAG | |
| 4D4 | --- --- --- --- --- --- --- --- --- --- --- --- --- --- | |
| | 30 | |
| | AGC CTT GTA CAC AGT AAT GGA AAC ACC TAT TTA CAT TGG TAC | |
| 4D4 | --- --- --- -A- --- --- --- --- --- --- --- --- --- --- | |
| | 40 50 | |
| | CTG CAG AAG CCA GGC CAG TCT CCA AAG CTC CTG ATC TAC AAA | |
| 4D4 | --- --- --- --- --- --A --- --- --- --- --- --- --- --- | |
| | 60 | |
| | GTT TCC AAC CGA TTT TCT GGG GTC CCA GAC AGG TTC AGT GGC | |
| 4D4 | --- --- --- --- --- --- --- --- --- --- --- --- --- --- | |
| | 70 | |
| | AGT GGA TCA GGG ACA GAT TTC ACA CTC AAG ATC AGC AGA GTG | |
| 4D4 | --- --- --- --- --- --- --- --- --- --- --- --- --- --- | |
| | 80 | |
| | GAG GCT GAG GAT CTG GCA GTT TAT TTC TGC TCT CAA AGT ACA | |
| 4D4 | --- --- --- --- --- -G- --- --- --- --- --- --- --- --- | |
| | CAT GTT CCT | |
| 4D4 | --- --- -G- | |
| J_{κ}1 | TGG ACG TTC GGT GGA GGC ACC AAG CTG GAA ATC AAA | SEQ. ID. NO.5 |
| 4D4 | --- --- --- --- --- --- --- --- --- --- --- --- | |

Although the invention herein has been described with reference to particular embodiments, it is to be understood that these embodiments are merely illustrative of various aspects of the invention. Thus, it is to be understood that numerous modifications may be made in the illustrative embodiments and other arrangements may be devised without departing from the spirit and scope of the invention.

We claim:

1. Monoclonal antibodies which bind to protective epitopes on serotype A, B, C and D strains of Cryptococcus neoformans, such protective epitopes containing acetyl groups in the polysaccharide of the epitopes.

2. Monoclonal antibodies according to claim 1 which have isotypes of IgM, IgA, IgG₁ or IgG₃.

3. Monoclonal antibodies according to claim 1 which have κ light chains.

4. Monoclonal antibodies according to claim 3 wherein the light chain is composed of V_{κ}5.1 and J_{κ}1.

5. Monoclonal antibodies according to claim 1 wherein the heavy chain variable region is composed of V_{H}7183-283, a diversity segment and J_{H}2.

6. Monoclonal antibodies according to claim 5 wherein the diversity segment consists of seven amino acids.

7. A method of making monoclonal antibodies which bind to protective epitopes on serotype A, B, C and D strains of Cryptococcus neoformans, such protective epitopes containing acetyl groups in the polysaccharide of the epitopes, which comprises:
(a) infecting animals with Cryptococcus neoformans serotype A strain organism;
(b) treating the infected animals with Amphotericin B intraperitoneally;
(c) assaying the sera of the infected animals by ELISA to determine which infected animals produced high serum titers of antibody to the Cryptococcus neoformans; and
(d) fusing spleen cells from high-titer animals and NSO myeloma cells to obtain monoclonal antibody-producing hybridomas.

8. Monoclonal antibodies produced by the method of claim 7.

9. A method of making monoclonal antibodies which bind to protective epitopes on serotype A, B, C and D strains of Cryptococcus neoformans, such protective epitopes containing acetyl groups in the polysaccharide of the epitopes, which comprises:
(a) immunizing animals with a glycoconjugate of Cryptococcus neoformans capsular polysaccharide and a protein carrier;
(b) assaying the sera of the immunized animals by ELISA to determine which animals produced high serum titers of antibody to the Cryptococcus neoformans; and
(c) fusing spleen cells from high-titer animals and NSO myeloma cells to obtain monoclonal antibody-producing hybridomas.

10. A method according to claim 9 wherein the protein carrier is tetanus toxoid.

11. Monoclonal antibodies produced by the method of claim 10.

12. A method of treating and preventing infection caused by serotype A, B, C and D strains of Cryptococcus neoformans which comprises administering an effective amount of monoclonal antibodies which bind to protective epitopes on serotype A, B, C and D strains of Cryptococcus neoformans, such protective epitopes containing acetyl groups in the polysaccharide of the epitopes.

13. A method of treating and preventing infection caused by serotype A, B, C and D strains of Cryptococcus neoformans which comprises administering an effective amount of monoclonal antibodies produced by the method of claim 7.

14. A method of treating and preventing infection caused by serotype A, B, C and D strains of Cryptococcus neoformans which comprises administering an effective amount of monoclonal antibodies produced by the method of claim 10.

15. A method of diminishing the level of serotype A, B, C and D strains of Cryptococcus neoformans polysaccharide circulating in body fluids which comprises administering an effective amount of monoclonal antibodies which bind to protective epitopes on serotype A, B, C and D strains of Cryptococcus neoformans, such protective epitopes containing acetyl groups in the polysaccharide of the epitopes.

16. A method of diminishing the level of serotype A, B, C and D strains of Cryptococcus neoformans polysaccharide circulating in body fluids which comprises administering an effective amount of monoclonal antibodies produced by the method of claim 7.

17. A method of diminishing the level of serotype A, B, C and D strains of Cryptococcus neoformans polysaccharide circulating in body fluids which comprises administering an effective amount of monoclonal antibodies produced by the method of claim 10.

18. Monoclonal antibodies which bind to protective epitopes on serotype D strain Cryptococcus neoformans.

19. Monoclonal antibodies according to claim 18 which have an isotype of IgM.

20. Monoclonal antibodies according to claim 18 which have λ light chains.

21. Monoclonal antibodies according to claim 20 wherein the light chain variable region is composed of V_{λ}2/J_{λ}2.

22. Monoclonal antibodies according to claim 18 wherein the heavy chain variable region is composed of V_{H}441, a diversity segment and J_{H}3.

23. Monoclonal antibodies according to claim 22 wherein the diversity segment consists of four amino acids.

24. A method of making monoclonal antibodies which bind to protective epitopes on serotype D strain Cryptococcus neoformans which comprises:
(a) infecting animals with Cryptococcus neoformans serotype D strain organism;
(b) treating the infected animals with Amphotericin B intraperitoneally;
(c) assaying the sera of the infected animals by ELISA to determine which infected animals produced high serum titers of antibody to the Cryptococcus neoformans; and
(d) fusing spleen cells from high-titer animals and NSO myeloma cells to obtain monoclonal antibody-producing hybridomas.

25. Monoclonal antibodies produced by the method of claim 24.

26. A method of treating and preventing infection caused by serotype D strain Cryptococcus neoformans which comprises administering an effective amount of monoclonal antibodies which bind to protective epitopes on serotype D strain Cryptococcus neoformans.

27. A method of treating and preventing infection caused by serotype D strain Cryptococcus neoformans which comprises administering an effective amount of monoclonal antibodies produced by the method of claim 24.

28. A method of diminishing the level of serotype D strain Cryptococcus neoformans polysaccharide circulating in body fluids which comprises administering an effective amount of monoclonal antibodies which bind to protective epitopes on serotype D strain Cryptococcus neoformans.

29. A method of diminishing the level of serotype D strain Cryptococcus neoformans polysaccharide circulating in body fluids which comprises administering an effective amount of monoclonal antibodies produced by the method of claim 24.
